(19) United States
(12) Patent Application Publication
(10) Pub. No.: US 2009/0149814 A1
(43) Pub. Date: Jun. 11, 2009

(54) CATHETER STRESS RELIEVER AND STABILIZER

(76) Inventors: Michelle Bailey, Hyde Park, UT (US); Penny Baker, Nibley, UT (US)

Correspondence Address: PATE PIERCE & BAIRD, 175 SOUTH MAIN STREET, SUITE 1250, SALT LAKE CITY, UT, 84111, US

(21) Appl. No.: 12/050,837

(22) Filed: Mar. 18, 2008

Related U.S. Application Data
(60) Provisional application No. 61/012,118, filed on Dec. 7, 2007.

(51) Int. Cl. A61M 25/02

(52) U.S. Cl. 604/180

(57) ABSTRACT

A stabilization and support for a catheter provides stress distribution for the loads otherwise imposed by the catheter interface hardware such as the base and hub components that may otherwise cause stresses in the skin and associated damage and sores. A mount is sized to support a catheter installation extending substantially tangentially to the surface of the skin of a subject, thus permitting stabilization of the catheter without exposing the skin to the sharp, highly stressful edges of catheter equipment. A transparent securement renders the catheter available for visual inspection of the condition of the catheter and the surrounding skin.

### BACKGROUND

1. The Field of the Invention

This invention relates to intravenous infusion and, more particularly, to novel systems and methods for catheter stabilization and protection.

2. The Background Art

Healthcare providers now commonly use forms of intravenous infusions to care for patients, often for administering blood, nutrients, or medication vital to the patient's health and safety. The use of catheters for these intravenous injections is now an industry standard due to ease of use, enabling providers to pierce the patient once every 48 to 72 hours, depending on variables such as condition of skin around the site, condition of insertion site, and the condition, age, and health of the patient.

A catheter also saves the patient from experiencing the pain of multiple needle insertions, which also limits exposure to external pathogens that can create complications or even death. Moreover, it also allows the provider to use fewer veins over a period of time, leaving open more options for the future, and limits the amount of damage done to a patient.

The majority of these complications are not caused by the catheters themselves, however, but rather by undesired movement of the catheters after installation. Catheter tip movement may stretch openings, irritate the vessel lining, triggering inflammation, thrombosis (blood clot), and eventual erosion of the venous wall. What is needed is an apparatus and method to stabilize a catheter to minimize secondary complications and damage.

### BRIEF SUMMARY OF THE INVENTION

In view of the foregoing, in accordance with the invention as embodied and broadly described herein, a method and apparatus are disclosed in one embodiment of the present invention as including an apparatus comprising an intravenous supply, needle, catheter, and a mount.

The intravenous supply may include a container for holding fluid such as physiological saline, a drug, both, or the like, and an adapter to conduct the fluid from the supply container to a line. The line is eventually connected to a hub. The hub may receive a shank of a catheter and be sealed thereagainst to receive and conduct the fluid therethrough.

In selected embodiments, the catheter may provide intravenous introduction of the fluid and contain a tubular portion placed into a vein and a shank or base secured to the tubular portion to connect to a hub. Accordingly, the base is sealed to the tubular portion, typically by being formed (e.g., molded) with the tubular portion to deliver fluid to the tubular portion.

In selected embodiments, the needle may be temporarily insertable through the tubular portion to render the tubular portion mechanically rigid during insertion into a vein of a subject by a user. In such embodiments, the needle may comprise a point extending beyond a distal end of the tubular portion to engage and penetrate a vein during insertion of the tubular portion. The needle may be used to reinforce the tubular portion and help it penetrate a vein.

In certain operational methods, the needle may be threaded through the tubular portion to render the tubular portion rigid and to expose a point of the needle for insertion into a vein of a subject. A healthcare provider may then select a site for insertion of the tubular portion into a vein and penetrate through the skin, inserting the needle and tubular portion of the catheter into the vein.

The provider may then withdraw the needle and position a mount between the skin of a user and at least one of the hub and the base. The mount may position the hub and base to extend substantially tangentially away from the site. Thus securing the catheter, hub, and mount proximate the penetration site, all move together with the patient's (subject's) skin without substantial relative motion therebetween.

In selected embodiments, the mount may be positioned between the skin of a subject and at least one of the shank and hub to distribute stresses imposed by them on the skin. In such an embodiment, the mount may comprise a lower surface provided with a layer of adhesive to secure the mount to the skin of the subject receiving the catheter.

Certain embodiments of a system may further include a securing member with a layer urging at least one of the base and hub towards the skin of a subject. The securing member may be transparent, rendering the catheter and surrounding skin of a subject visible for visual inspection by a user. For example, in one embodiment a layer of transparent film, permeable, semi-permeable, or impermeable, may seal the site. A semi-permeable, breathable membrane, flexible to conform to the system may reduce chances of infection and avoid moisture accumulation. Transparent film secured by a border or tape may be used to cover both the mount and catheter, urging both towards the skin and securing them to the subject.

In selected embodiments, the mount may be designed to keep at least one of the base (shank) and hub away from contact with the skin. In such an embodiment, the shape of the mount may distribute the load imposed by the catheter on the upper surface thereof across the lower surface of the mount. That is, the mount is shaped to distribute stresses imposed upon it to the skin at a reduced value over an increased area of the skin.

In certain embodiments, it may be desirable to use a polymer to manufacture the mount. The polymer may be expanded to form a foam to make it softer and lighter, causing it to become more comfortable when held flush against the skin. In such embodiments, the expanded polymer may be a closed cell foam, making it non-absorbent, an environment less conductive to moisture, soiling, or bacteria growth.

In certain embodiments, the shape of the mount may be shaped to provide a relief region to receive at least one of the base and hub thereinto to stabilize the catheter against movement with respect to the mount and the skin of the subject.

For example, the mount stabilizes the catheter and secures it to the skin so that both will experience the same movement, resisting any tendency of the tip of the catheter toward twisting, kinking, bending, wiggling laterally, sliding axially, or rubbing against the vein of the subject. In such embodiments, the mount may orient at least one of the base and hub to extend substantially tangentially away from the skull. Accordingly, this firmly mounts the hub and shank, yet reduces dramatically the direct pressure from either the shank or the hub against the skin.

### BRIEF DESCRIPTION OF THE DRAWINGS

The foregoing features of the present invention will become more fully apparent from the following description and appended claims, taken in conjunction with the accompanying drawings. Understanding that these drawings depict only typical embodiments of the invention and are, therefore, not to be considered limiting of its scope, the invention will be described with additional specificity and detail through use of the accompanying drawings in which:

FIG. 1 is perspective view of one embodiment of an apparatus in accordance with the invention in an installed circumstance;

FIG. 2 is an exploded, perspective view of alternative embodiments of a mount in accordance with the invention

FIG. 3 is a bottom perspective view of a mount in accordance with the invention, including an adhesive layer and a protective peel-off cover;

FIG. 4 is a side elevation view of one embodiment of an installation of a catheter using a mount in accordance with the invention;

FIG. 5 is a side elevation view of an alternative embodiment of an installation relying on the shank being in contact with the mount in accordance with the invention;

FIG. 6 is a perspective view of an apparatus in accordance with the invention installed under a clear, semi-permeable membrane cover;

FIG. 7 is a perspective view of one embodiment of a kit containing key elements for installing a catheter on a mount in accordance with the invention;

FIG. 8 is a schematic block diagram of one embodiment of a method in accordance with the invention for implementing a catheter installation using a tangential mounting block; and

FIG. 9 is a schematic block diagram of a kit containing components for installing a catheter on a mounting block in accordance with the invention.

### DETAILED DESCRIPTION OF THE PREFERRED EMBODIMENTS

It will be readily understood that the components of the present invention, as generally described and illustrated in the drawings herein, could be arranged and designed in a wide variety of different configurations. Thus, the following more detailed description of the embodiments of the system and method of the present invention, as represented in the drawings, is not intended to limit the scope of the invention, as claimed, but is merely representative of various embodiments of the invention. The illustrated embodiments of the invention will be best understood by reference to the drawings, wherein like parts are designated by like numerals throughout.

Healthcare providers now commonly use forms of intravenous infusions to care for patients, for example, for administering blood, nutrients, or medication, vital to the patient's health and safety. The use of catheters for these intravenous injections is now an industry standard due to its ease of use, enabling providers to pierce the patient once every 48 to 72 hours, depending on variables such as conditions around site.

For use with children, catheters are commonly inserted in the arms, hands, or feet. However, these areas tend to be exposed to constant movement, especially if a small child is scared, causing the child to cry, kick, and even thrash about. Even should the child prove cooperative, many other challenges meet the healthcare provider due to physiological differences in small children.

Physically, a child has smaller veins than those of an adult and layers of subcutaneous fat (layers of fat right below the skin) makes seeing and palpating veins (examination by touch) very difficult. Additionally, children who are dehydrated, septic (contain pathogenic organisms), or incur trauma pose an even greater challenge in obtaining access.

However, because these areas tend to be exposed to constant movement and physiological differences in a child, alternative insertion locations have been used, most notably the scalp. The benefits of using the scalp as an insertion site are that the veins are superficial, making them easier to see and palpate. There are no valves to contend with, decreasing chances of inflicting damage to the patient. Furthermore, the head does not need to be immobilized because its range of activity is rather limited, subjecting the child to less emotional trauma by not restricting movement in any of his extremities. Some possible sites include the superficial temporal, posterior auricular frontal, occipital, and supraorbital veins.

Some common complications of intravenous catheters include infiltration (e.g., IV fluids and medication flowing into surround tissue), extravasation (e.g., a particularly irritating drug infiltrating surrounding tissue), phlebitis (e.g., inflammation of an interior surface of a vein), hematoma (e.g., bruising), IV-related infections, occlusion (obstruction of the vein), dislodgement (e.g., movement of the catheter or even the vein from normal position), tissue sloughing (e.g., formation of dead tissue), and various systemic complications (e.g., invasion of the blood by microorganisms), some of which can be fatal.

Current methods of catheter stabilization include the use of tape and gauze or cotton to stabilize and protect the catheter and insertion site. Additional tape is further used should the first layer of adhesive prove insufficient to adequately stabilize the catheter. However, these layers of tape create unnecessary pressure on the skin, creating discomfort and can lead to symptoms such as itching, rash, or other allergic reactions. Furthermore, the pressure forces the hard and oftentimes sharp base of the catheter or of a hub against the skin, causing irritation and further discomfort to the patient. Layers of tape also decrease the visibility of the insertion site, impairing the ability of the healthcare provider to assess the health and condition of the site, disabling them from diagnosing problems in its early stages. Moreover, the methods are often satisfactory only for patients who are able to understand and comply with instructions, stay still, and are healthy. However, this creates a problem for children, especially those under the age of one, because they are not able to comprehend anything and move around with alarming frequency.

Due to the sensitivity of the sites associated with the skull, it is best for the needle and catheter to extend tangentially from the skull, preventing damaging the skull and other vulnerable areas on the scalp. Current practices involve the use of cotton or gauze underneath the catheter as a pillow. However, both of those materials are highly absorbent, making any absorption from leakage or otherwise a breeding ground for bacteria and other pathogens. They are also very soft and will collapse under pressure, allowing the catheter shank and hub to continue rubbing against and irritating the skin. Furthermore, they, especially cotton balls, will interfere with adhesives, thus decreasing the stability of the entire system and increasing the risk of more complications.

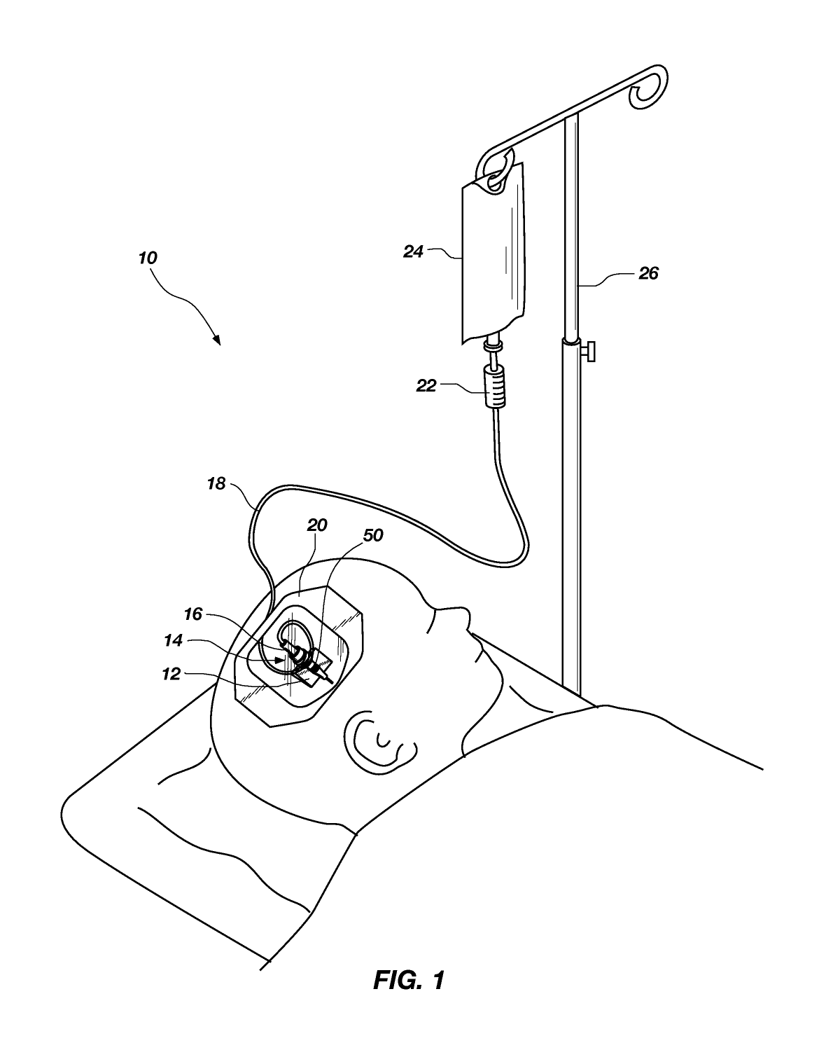

Referring to FIG. 1, a system 10 may include a mount 12 or block 12 for mounting a catheter 14. Typically, a catheter 14 may be connected to a hub 16 fed by a line 18. The line 18 may carry a medicament, a hydration solution, or the like. For example, physiological saline containing water, sugar, and a quantity of salts may be used as a hydration material. Likewise, a particular medicament may be installed as a drip feeding into a metering device 22 at a particular rate. Accordingly, a hydration material may be passed through the line 18, in conjunction with a medicament such as a drug, an antibiotic, a necessary chemical, or the like.

A cover 20 may provide protection against infection, exposure to sources of germs, bacteria, and the like. In one embodiment, the cover 20 may be formed of a semi-permeable membrane in order to pass vapor but not liquid. Accordingly, the cover 20 may be installed in such a way as to seal off the area containing the catheter 14, while not preventing evaporation. Thus, the region under the cover may remain clear and not clouded by vapor droplets, and the like, obstructing vision.

In one embodiment, the cover 20 may be formed of opaque material. However, it is good practice to be able to visually inspect the site of installation of the catheter 14. Accordingly it may be appropriate to provide a cover 20 having a substantially transparent view in order to permit ready, visual inspection without opening the cover.

For example, it is a common practice to add tape to a catheter 14 in order to further support the catheter 14 near the skin of a subject. However, such approaches tend to both introduce bacteria from non-sterile sources as well as adding additional pressure to the catheter 14, hub 16, or both, increasing the concern with secondary harm to a subject. Accordingly, in one embodiment, the mount 12 under the catheter 14, hub 16, or both may distribute stress from the comparatively sharper edges of the catheter 14 and hub 16, thus providing a lower and distributed stress at the skin of a user.

Typically, a line 18 may be fed from a supply 24. A supply 24 may be a bottle, bag, or the like as known in the art. The supply 24 may be suspended at some height above the site of a catheter 14 in order to provide a certain “head” or pressure to maintain flow. Typically, a stand 26 may support a supply 24 in accordance with the invention.

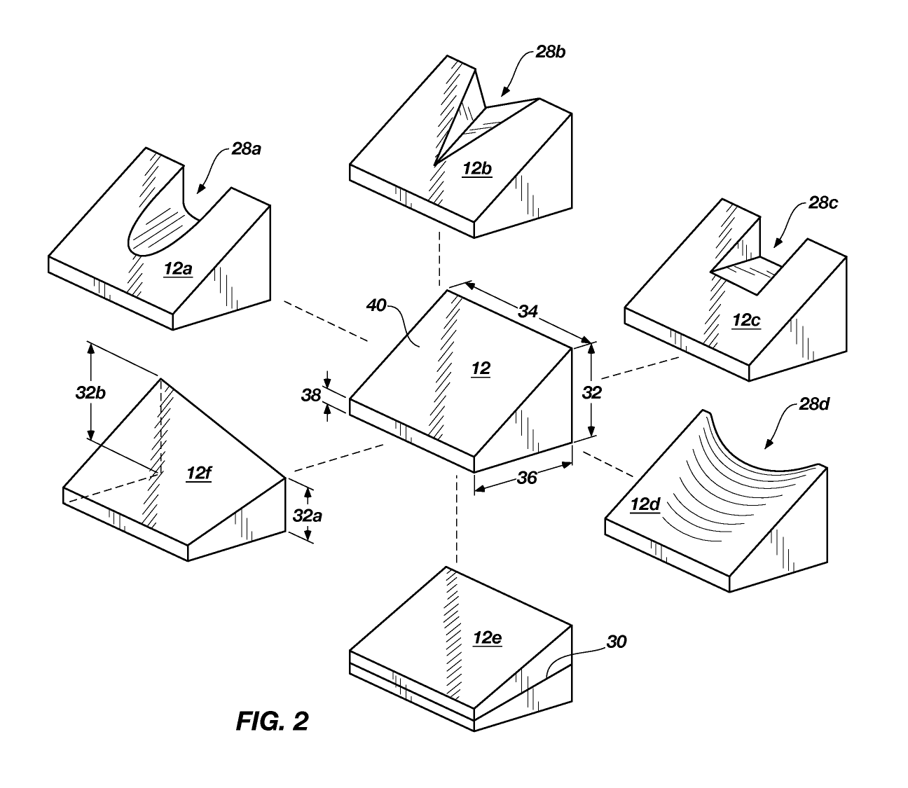

Referring to FIG. 2, a mount 12 may be formed to be shaped like a wedge. In certain embodiments, the mount 12 may be a block, a mat, or the like. However, it has been found effective to select a size and dimension for the block 12 or mount 12 to interface between the skin of a user and the catheter in a somewhat tangential relationship. For example, in pediatric catherization, the skull is a likely location for catheters. However, the curvature of the skull necessarily leaves the shank of a catheter 14 extending at a tangent to the skull. Accordingly, a mount 12 may be interposed in order to stabilize the catheter 14 with respect to the skull of an infant.

In FIG. 2, various alternative embodiments are illustrated. For example, each of the mounts 12a-12f is shown in a slightly different configuration. In one embodiment, a relief 28 may be provided. The relief 28 may be shaped semi-circularly, as a slight depression providing the mount 12 a hollow for the receipt of the catheter 14, hub 16, or both therein.

In alternative embodiments, a v-shaped relief may be imposed on an upper portion of the mount 12 to receive the larger, rounded portion of the catheter 14 (e.g., shank 50), the hub 16, or the like. Alternatively, the relief 28c may be formed in a rectangular form.

In general, a mount 12 may be formed to have an interface 30 for securing multiple mounts 12 together. For example, in the embodiment of the mount 12e, the interface 30 may represent an adhesive joint between two mounts 12e. Nevertheless, it has been found effective in the circumstance of dealing with infants to have a mount 12 ready to install with a minimum of delay. Accordingly, stacking of mounts 12e may take additional time that need not be taken.

For example, the mount 12 may be formed to have a height 32 selected to fill the tangent space between a catheter 14, hub 16, or both beside the skull of a subject. Meanwhile, the mount 12 may have a length 34 selected to readily receive the width of a catheter shank 50 or hub 16. The width 34 may also be selected to be sufficiently wide that the contact pressure of the cover 20 against the upper corners of the surface 40 may secure the catheter 14 and hub 16 therebetween, while also securing the catheter 14 and hub 16 against the surface 40.

A width 36 of a mount 12 may be selected to correspond to the length of a shank 50 of a catheter 14. For example, the offset 38 near the toe of the mount 12 of FIG. 2 is selected to provide a distance of clearance between the shank 50 of a catheter 14 and the skin of a user. Accordingly, the distal or narrow end of the shank 50 need not be placed in contact with the skin of a user. Typically, the height 32, length 34, and width 36, as well as the offset height 38 may be selected in order to optimize the performance of the mount 12.

For example, the height 32 may be selected to be from about ⅛ inch to about ½ inch in height. Typically, it has been found that a target distance 32 of about ⅜ inch is very satisfactory. If the width 36 is selected to be about a half an inch, then the length of the shank 50 may be readily accommodated on the top surface 40 of the mount 12.

Likewise, the length 34 may be from about ⅜ inch to about 1 inch in length. However, it has been found that a target length 34 of about ¾ inch provides sufficient space on either side of a hub 16 or catheter shank 50 in order to engage tape, a cover 20, or the like. The width 36 is typically better served by being at least long enough to support a shank 50 and hub 16 of a catheter 14. Thus, although a width 36 of one inch or more is completely tractable, it may be limited to a distance that may be readily covered with a cover 20. Accordingly, it is recommended that the width 36 not dominate the hub 16 and shank 50. Accordingly, dimensions 32, 34, 36, 38 sized to be slightly larger than those corresponding to the hub 16 and shank 50 appear to provide the best utility and the least interference for the mount 12.

In one embodiment, target distances of a width 36 of ½ inch, a height 32 of about ⅜ inch, a length 34 of about ¾ inch with about a 1/16 height offset 38 have been found effective as target distances. A distance within reasonable tolerances of these may be provided in a mount 12 fabricated by suitable manufacturing processes.

The mount 12, in general, may be fabricated in any of several suitable manufacturing processes. In one presently contemplated embodiment, the material of the mount 12 may be an expanded polymer selected from various hypoallergenic polymers. In certain embodiments, the polymer may even be a styrene. However, softer resilience may be preferred, such as is found in urethanes, expanded elastomers, “gels,” and the like.

The structural material may be treated, doped, embedded, or otherwise prepared to resist germs, bacteria, viruses, or the like. A treatment may be selected for the surface or to be disposed throughout the mount 12. The treatment may be selected from a variety of germicides, antiseptics, antibiotics, anti-virals, and the like. Carriers and active ingredients may be selected to be most effective against contemplated organisms of typical or greatest concern.

In certain embodiments, the expanded polymer may be formed as a closed-cell foam structure. Such structures may be easily extruded to have the wedge-shaped cross section illustrated in the mount 12 of FIG. 2. The various versions of relief 28 provided to receive the shank 50 or hub 16 maybe formed typically by molding the mount 12, or by a secondary removal process (e.g., drilling, cutting, etc.) In order to obtain the relief portion.

In one embodiment, the mount 12 may simply be cut at each end from a long extruded cross section. This makes the manufacturing process very fast, economical, and readily adaptable to mass production.

The embodiment of the mount 12f is provided with a height 32a that is comparatively shallower at one end, with an alternative height 32b that is potentially higher at the opposite end. Such an embodiment provides an inherent stability in that the catheter 14 or hub 16 secured thereto may be said to have a shallow or a steep angle with respect to a subject. Nevertheless, since the catheter 14 will typically be secured by a cover 20, tape, or the like, the value of the stress distribution of the base 12 or mount 12 is still available. Similarly, the upper corners of the mount 12f are still located where they can engage tape, a cover 20, or the like to secure the catheter 14 or hub 16 therebetween. Nevertheless, the basic mount 12 has been found to quickly and effectively, provide the proper angle for pediatric installations.

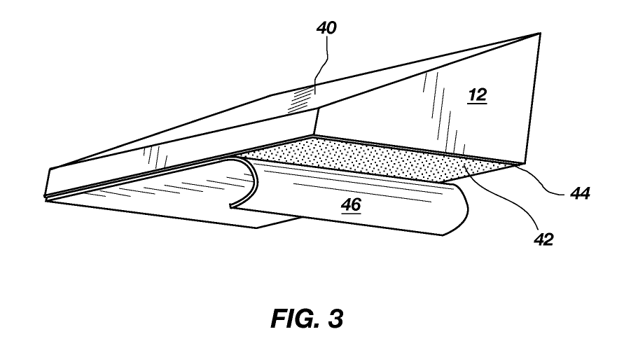

Referring to FIG. 3, in one embodiment a mount may have an adhesive layer 44 attached to a bottom surface 42 thereof. Meanwhile, a peel-off protective layer 46 may maintain the adhesive layer 44 clean and ready for adhering to a subject. In certain embodiments, the adhesive layer 44 may be selected to secure to the skin of a user, in spite of intervening wisps of hair that may interfere.

In some embodiments, preparation of a patient may include shaving an area for adherence of the adhesive layer 44 against the skin. However, it has been found that, in general, the adhesive layer 44 may be secured against the skin of a user and may be suitably removed without pain by selection of the proper adhesive quality of the layer 44.

As a practical matter, the adhesive layer 44 need not be responsible for the entire maintenance of position. As it turns out, the adhesive layer 44 is a substantial aid to resisting any sliding of a catheter 14 laterally (e.g. in any direction at right angles to the direction of insertion). Likewise, any axial dislocation along the direction of the catheter 14 is to be avoided. Accordingly, securement by an adhesive layer 44 against the skin of a user provides a comparatively rigid base. Therefore a cover 20 or tape over the top of the mount 12, capturing the catheter 14 and hub 16 thereabove provides an additional degree of stability.

For example, otherwise, if a catheter 14 or hub 16 were simply located under a piece of tape, and could still slide with respect to the skin of a user, then the contact by tape with an upper extremity of the shank 50 or hub 16 would not necessarily stabilize the catheter 14. By contrast, a mount 12 in accordance with the invention, by providing an adhesive layer 44 fixed to the skin of a user, tends to fix the bottom edge of a catheter 14 and hub 16 thereagainst. Clear tape, a transparent cover 20, or the like secures the upper reaches of a catheter 14 and hub 16 thereagainst. Accordingly, rolling, sliding, and other dislocations of a catheter 14 are much less likely and are resisted by substantially greater forces than provided in prior art installation methods.

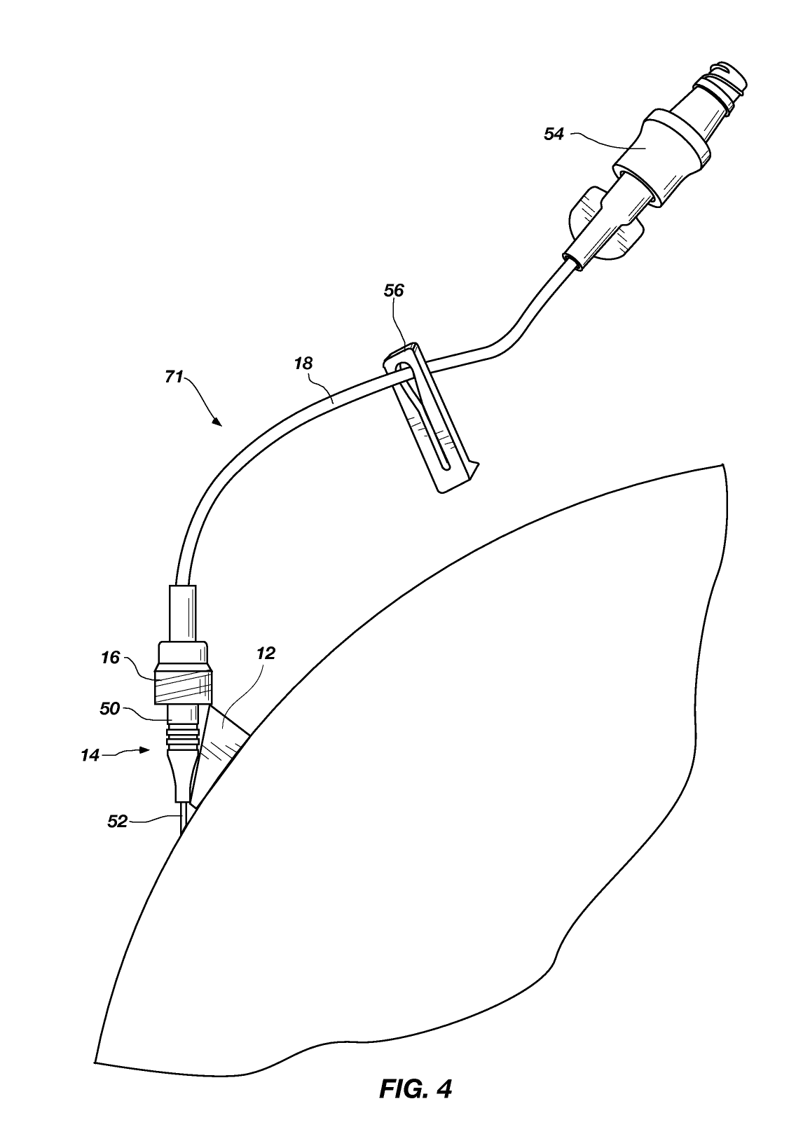

Referring to FIG. 4, an installation of a catheter 14 in accordance with the invention may typically be accomplished in one of two principal ways. The mount 12 may be secured by the adhesive layer 44 against the skin of a user. However, either the shank 50, the hub 16, or both may be set on the upper surface 40 of the mount 12.

For example, in the illustration of FIG. 4, the hub 16 itself is set on the upper surface 40 of the mount 12. The shank 50 then extends towards a subject. Meanwhile, a tube portion 52 of the catheter has been inserted under the skin and into a vein. The mount 12 assures that the angle between the shank 50 and the vein of a subject receiving the tube 52 of a catheter 14 will not present such a severe angle as to kink the tube 52, thus occluding its interior.

Meanwhile, a line 18 may form part of an assembly 71 beginning with the hub 16 passing through the line 18 and including a clamp 56. The clamp 56 may be used to change the rate of flow of liquids through the line 18. Likewise, the clamp 56 may be used to completely shut off the line 18 during installation or at other times. Meanwhile, an adapter 54 secures to the remaining assembly feeding from the supply 24.

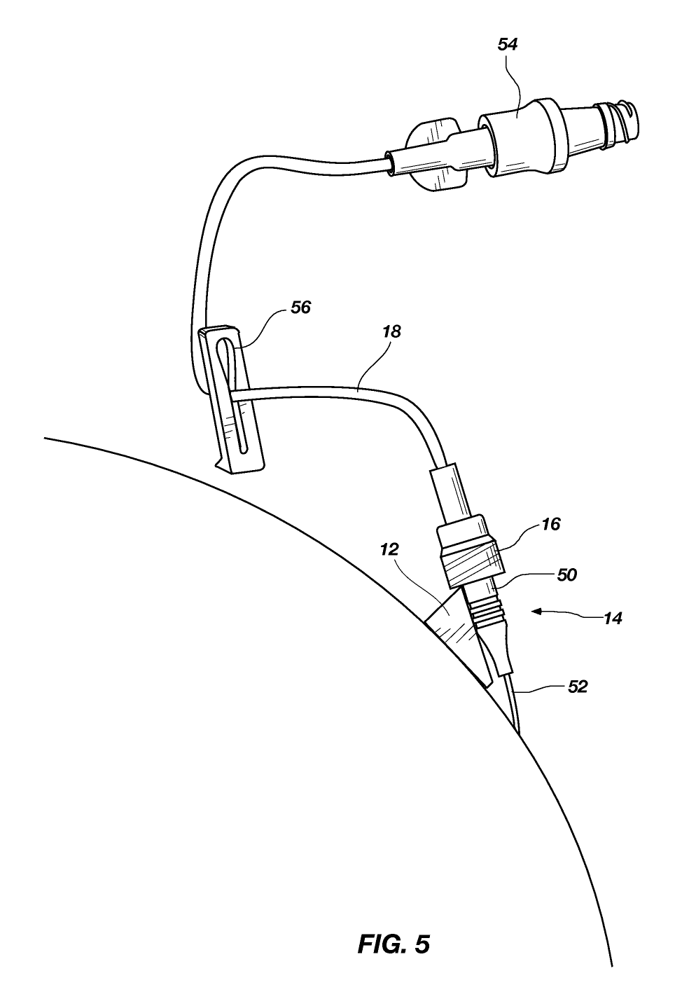

Referring to FIG. 5, an alternative installation illustrates the mount 12 secured to the skin of a user and receiving the shank 50 on the upper surface 40 of the mount 12. Meanwhile the hub 14 is set completely behind the upper edge of the mount 12. In one embodiment of an apparatus 10 in accordance with the invention, the length of the shank 50 is such that the mount 12 will completely support the shank 50 away from the skin of a user.

Meanwhile, the shank may 50 extend just beyond the lower toe edge of the mount 12, into the offset height 38. Accordingly, the narrowest portion of the shank 50 will typically not impose any substantial stress on the skin of a user. The mount 12 may be sized such that the catheter tube 52 itself actually extends along the upper surface 40 of the mount 12. However, it is not been found necessary to do this in experiments with the apparatus 10.

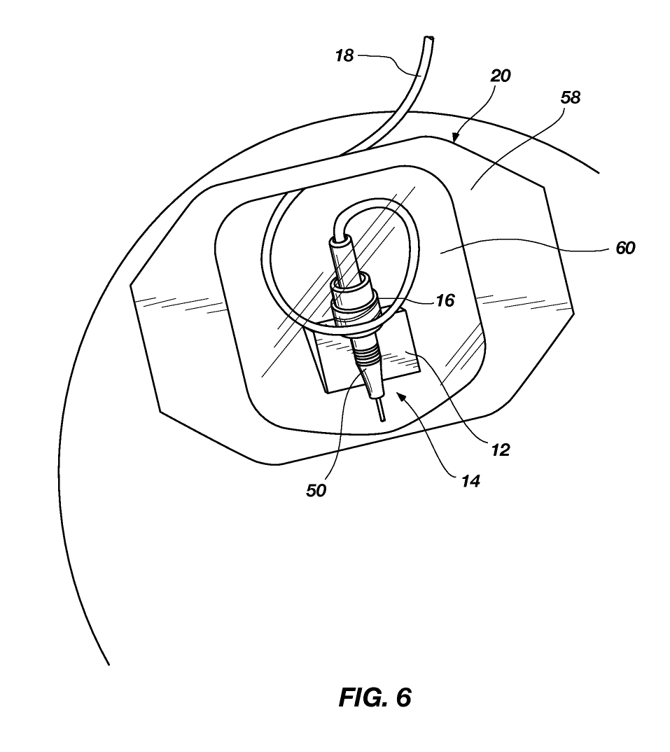

Referring to FIG. 6, a typical installation may include the mount 12 secured by an adhesive layer 44 to the skin of a user, and a catheter 14 installed in a vein of a subject. Meanwhile, the shank 50 extends along the upper face 40 of the mount 12. In one embodiment, in order to provide some slack against an accidental pull on the line 18, the line 18 may actually be coiled around and on top of the shank 50. A cover 20 formed to have a border 58 sealable by a contained adhesive may provide both securement of the catheter 14 and hub 16 against the mount 12, but also seal against contact with bed clothes, touching, exposure to air or moisture, or the like.

Typically, a clear membrane 60 may form a window through the cover 20 permitting visible inspection. In certain embodiments, a conformal membrane 60 permits a certain amount of stretching to impose a load on the hub 16 and catheter 14 to secure them against the mount 12. A clear adhesive tape may also be used as one layer of securement. However, it has been found that maintaining clear the region around the catheter, particularly near the entrance of the tube portion 52 into the skin is extremely valuable as a visible inspection mechanism to assure that no complications are occurring due to shifting, movement, dislocation, bacteria, or the like.

One other advantage of laying the line 18 over the shank 50 over the catheter 14 is that the line 18 itself provides a member to apply force on the shank 50 to secure it against the upper face 40 of the mount 12. This is not essential, but may provide a certain degree of additional stability.

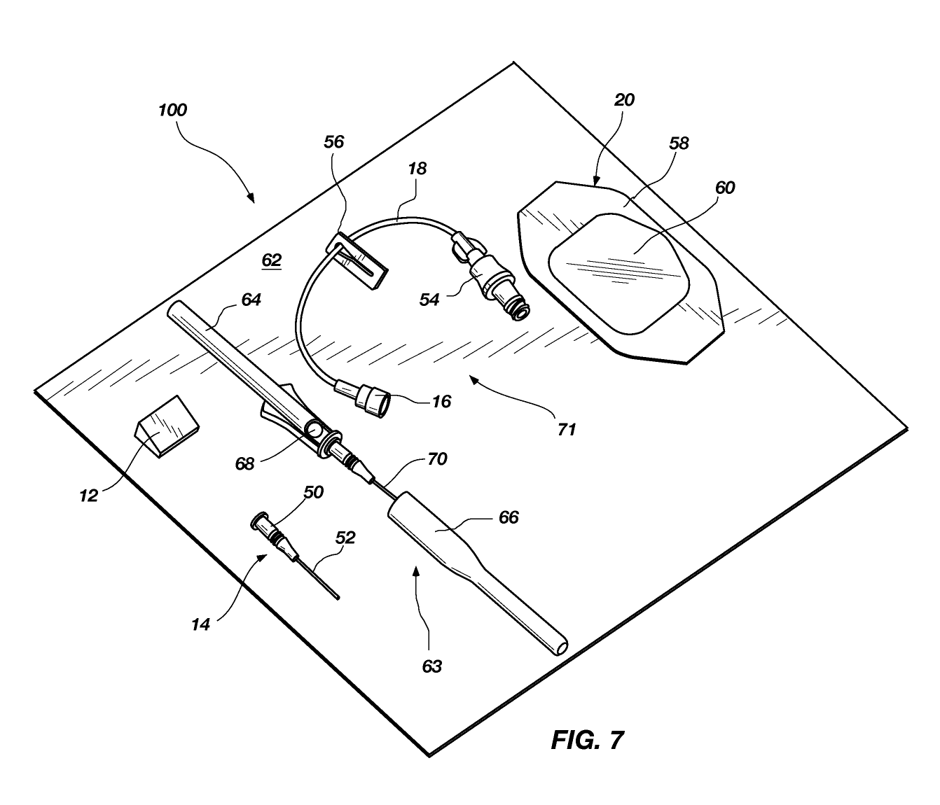

Referring to FIG. 7, a kit 100 for implementing an apparatus 10 in accordance with the invention may include a towel 62 providing a staging space for the components in accordance with the invention. In one embodiment, a needle assembly 63 may include a handle 64, as well as a cover portion 66. A release 68 may operate to retract the needle 70 back into the handle 64 after use.

Typically, the mount 12 may be installed under the shank 50, the hub 16, or both after insertion of the catheter tube 52 into a vein of a user. Typically, the needle 70 is threaded through the shank 50 and the catheter tube 52 exposing the point of the needle 70 beyond the end of the tube 52. Accordingly, the needle 70 may then penetrate the flesh and the selected vein for insertion of the catheter tube 52. The needle 70 may then be withdrawn and the release 68 activated to draw the needle 70 back into the handle 64. Thus, no sharp ends will further be exposed.

Typically, the hub 16 may then be secured with both a friction and rotary (e.g., threaded) fit between the shank 50 and the hub 16. The clamp 56 may then be opened after the adapter 54 has been connected to the supply 24. Typically, the connection between the hub 16 and the 50 may wait until after the entire line 18 from the supply 24 has been filled with liquid down through the exit of the hub 16. Accordingly, no bubbles will be introduced from the supply line 18 to the catheter 14.

A cover 20 may be sized to have a suitable border 58 provided with an adhesive for securing to the skin around the installation site. Meanwhile, the membrane 60 may be provided to be of sufficient strength that it will not tear or otherwise be compromised by any of the jutting edges of the hub 16, shank 50, or the like.

Typically, clear, sterile, adhesive tape may be used to secure the shank 50 against the mount 12. Likewise, tape may secure the hub 16 to the mount 12. This may be done while still leaving plenty of visible space near the tube 52 in order to view through the membrane 60 the area surrounding the insertion site of the catheter 14.

The interface assembly 71 may typically include about 6 inches of the line 18. The length is somewhat arbitrary. However, in one embodiment, the assembly 71 provides the necessary hub 16 end adapter 54 to connect between the shank 50 of the catheter 14, and the fixtures associated with the supply 24. In certain embodiments, the hub 16 may be connected directly to a line 18 integrated with the supply 24.

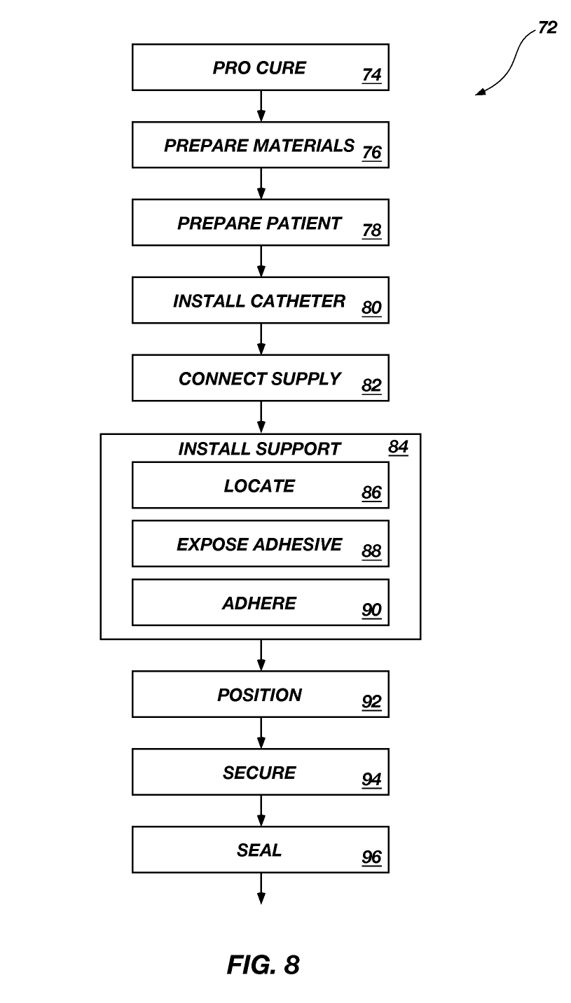

Referring to FIG. 8, a process 72 for installing an apparatus 10 in accordance with the invention may include procuring 74 the components provided with the kit 100. These components may include conventional supplies as well. Following procuring 74, preparing 76 the materials may involve purging air from lines, setting up the supply 24, laying out the catheter 14, and assembling the needle 70 into the shank 50 and tube 52 of the catheter 14.

Since the supply 24 will typically be elevated, securing a clamp 56 to prevent weeping of the supply fluid thought the hub 16 prior to installation may be appropriate. Preparing 78 the patient may involve shaving an area of the head. However, this is entirely optional and is not necessary. In some instances, the adhesive layer 44 of the mount 12 may stick to hair. Care may be taken to either shave or remove hair that may adhere to the adhesive layer 44 or extra care in removal may simply obviate the need for shaving.

Typically, the step of preparing 78 a patient may involve swabbing an area with cleansers, disinfectants, and the like. That is, germicides, antiseptics or the like may be used to prepare a site to assure that injection of the needle 70 under the skin does not introduce bacteria under the skin

Installing 80 a catheter 14 typically involves injecting the needle 70 with the tube 52 of the catheter sheathing around the needle 70 following the point of the needle to the targeted vein. The shank 50 is held while the needle is withdrawn therefrom. Accordingly, the catheter tube 52 remains in place in the vein extending out through the flesh through the skin penetration. Accordingly, the release 68 may be tripped in order to withdraw the needle 70 into the handle 64 where the cover 66 and handle 64 may both be thrown away.

Connecting 82 the supply 24 to the catheter 14 may be done before or after installing 84 the mount 12. However, in one embodiment, the adapter 54 may be secured to the supply 24 and any intervening lines 18 between the supply 24 and the adapter 54. Meanwhile, the extension line 18 extending from the adapter 54 to the hub 16 may be purged of any air by opening the clamp 56 to run fluid into the line 18 and hub 16. Typically, the fitting for the shank 50 will include both a friction fit and a rotary, threaded securement to the hub 16.

Installing 84 the support 12 or mount 12 may involve locating 86 the mount 12 at a suitable place under the shank 50, hub 16, or both. Exposing 88 the adhesive layer on the bottom surface 42 of the mount 12 may be accomplished by removing the peel-off layer 46 protecting it. The adhesive layer 44 may then be adhered 90 to the skin of a subject, thus positioning and securing the mount 12 under the catheter 14.

Positioning 92 the hub 16 and catheter 14 laterally on the mount 12 may be optional. For example, the catheter 14 may be moved slightly to be oriented towards one end of the other of the mount 12. Typically, the catheter shank 50 may be positioned such that it is not applying pressure to the skin. Likewise, the tube 52 should provide a smooth transition of curvature from the shank 50 into the skin penetration in order to not provide any kinking or other blocking of the catheter tube 52.

Securing 94 may involve taping the shank 50 down against the skin of a user, and against the mount 12. Typically, such pressure exerted by tape holding down a shank 14 and hub 16 may cause the abrupt edges thereof to create undue pressure on the skin. Due to the intervening location of the mount 12, such stresses will be distributed much more evenly and over a much larger region, mainly the bottom surface 42 of the mount 12.

Typically, the region of the penetration of the tubing 52 of the catheter 14 of the skin should all be left visible for inspection. Meanwhile transparent tape may actually secure by appropriate selection of the length 34 of the mount 12, the shank 50 between the upper corners of the top surface 40 of the mount 12. Accordingly, the shank 50 may be stabilized against the mount 12 and the mount 12 may be secured to the skin by an adhesive layer 40. Meanwhile, transparent tape may also further secure the mount 12 and the shank 50 with respect to the skin of a subject.

Sealing 96 is preferably accomplished by a cover 20 having a transparent membrane 10. Accordingly, the visibility of the tube 52 and its skin penetration may remain to support visual inspection. Meanwhile, the membrane 10 may stretch or wrap around the catheter shank 50, hub 16, and mount 12.

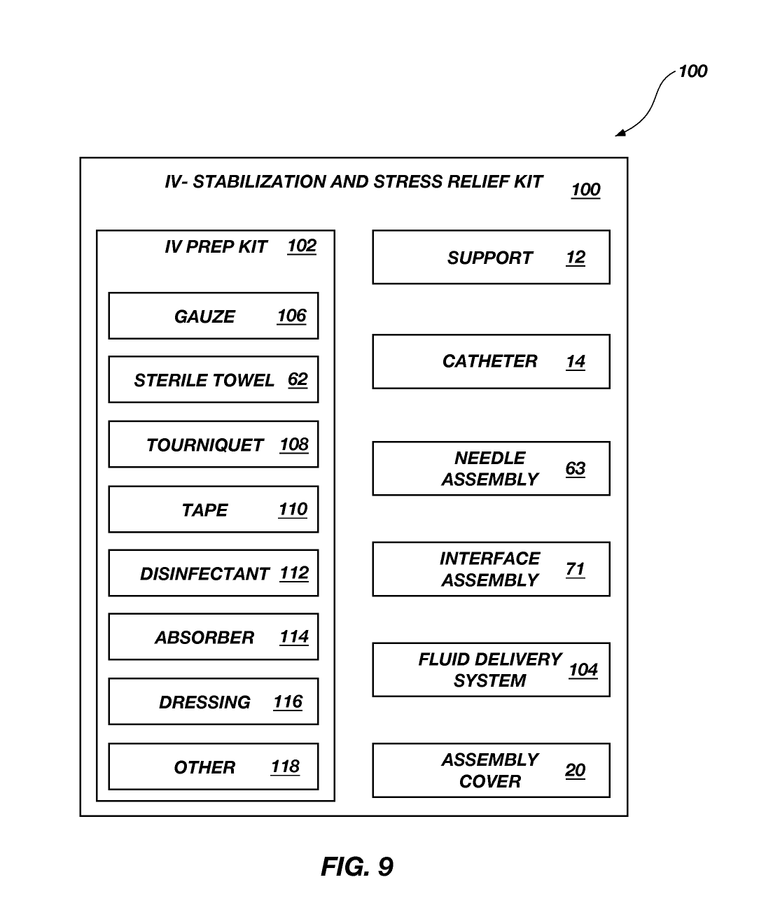

Referring to FIG. 9, a kit 100 may include both a preparation kit 102 as well as several other constituents in accordance with the invention. For example, the preparation kit 102 may involve materials for preparation of the site. However, the support 12 along with a catheter 14, a needle assembly 63, and interface assembly 71 to connect between the catheter 14 and the supply 24 may provide both the necessary connection components 16, 54, as well as a controlling clamp 56 on the segment of line 18.

A fluid delivery system 104 may include the supply 104 and any intervening lines 18 or other adapters necessary to connect to the assembly 71. Likewise, a stand 26 for elevating the supply 24 in order to generate the driving pressure head required may be part of the fluid delivery system 104.

An assembly cover 20 may be included in the kit 100. Typically this will be larger than a conventional dressing that would otherwise be used with such systems. The cover 20 may be selected to provide protection, visibility, and sealing, as well as mechanical stabilization in some instances.

Typically, the preparation kit 102 may include gauze or other materials suitably absorbent for particular tasks. However, gauze 106, cotton, and the like will typically not be recommended for leaving on site in an apparatus and method in accordance with the invention. For example, the stress relief of the base 12 and the need for maintaining cleanliness may obviate any need for any absorbent materials that may maintain liquids in the vicinity of the catheter 14.

Typically, a towel 62 prepared to be sterile in order to lay out the components of the apparatus 10 may be provided along with a tourniquet 108. Tourniquets 108 may typically be used to cause veins to distend in order to be more easily located and tapped by a catheter 14. Even though an apparatus 10 in accordance with the invention seems to be almost an imperative necessity for cerebral locations of catheters 14 in infants, the mount 12 may also be used in other physical location, such as arms, legs, and the like where catheters 14 may be installed.

Typically, a supply of sterile, transparent, adhesive tape 110 may be included in the kit 102. Tape 110 may typically be a sterilized supply, of a suitably transparent plastic in order to provide visible inspection thereunder. Typically, a disinfectant 112 may be employed to prepare an area for insertion of a catheter 14. Likewise, an absorber 114 for applying or removing any excess liquids, drips, or the like may be provided.

A dressing 116 may be replaced by the cover 20. For example, typically, a dressing may provide various functionalities. Typically, a dressing will provide protection against abrasion, touching, scratching, damage to the underlying constituents, protection against water, protection against air, protection against bacterial contact, and the like. Typically, a dressing 116 needs to be functionally inadequate for transparently covering the mount 12 and the entire apparatus associated therewith. Typically, it has been found that the presence of any absorbent materials under the cover 20 is inadvisable as it provides a breeding ground for bacteria. Other constituents 118, such as conventional antiseptic preparations, may be included in the preparation kit 102.

In summary, it may be seen that an apparatus in accordance with the invention may provide a mount formed of a closed-cell foam. Such materials are available that are latex free and of medically approved elastomers. Typically, the adhesive should be selected to be sufficiently strong to connect to the product, and to secure reliably to the skin of a user, yet have a peel strength selected to be gentle enough to be removed from skin, and removed from hair without undue discomfort.

Typically, a close-cell foam will provide no absorption of liquids, and thus no hosting for bacteria. Thus, the mount 12 may be manufactured to be sterile, and may be included in a kit 100 for implementing a method in accordance with the invention. By selecting a constant cross-section for the mount 12, manufacturing difficulty may be minimized, as the closed-cell elastomeric foam is extruded.

Certain coatings, including antibacterial materials, and the like may be provided for the mount, as well as various surface treatments in order to render the mount 12 antiseptic. Optionally, an adhesive or gripping material on the upper surface 40 may protect against sliding of the shank 50 or hub 16 with respect thereto.

Typically, the material properties of the mount may be selected to provide sufficient softness that the load placed on the shank 50 and hub 16 is distributed across the skin. According to St. Venant's principle, stress will be distributed along principal stress lines from a point of application. Accordingly, the height 32 of the mount 12 may be selected to evenly distribute the stress from the shank 50, hub 16, or both across the entire lower surface 42 of the mount 12.

The angle formed by the upper surface 40 and the lower surface 42 and their respective widths 36 may be selected to provide support along the shank 50, hub 16, or both. Due to the non-absorbent nature of the closed-cell foam of the elastomer forming the mount 12, a decrease may be realized site rotations due to soiling, secondary complications, and the like, which effects may thus be minimized, neutralized, or both.

In certain embodiments, the mount 12 may be placed proximal or distal to the hub 16. Notwithstanding the additional height 32 imposed by the mount, the actual bulk of dressings is reduced. For example, mounts of cotton, gauze, tape, and the like typical of prior art installation of catheters 14 are gone. Instead, close, secure adhesive connections are appropriately located, and securement to the skin of a subject is reliable. Accordingly, the use of gauze and cotton packing with its obfuscation of the site and its absorption of moisture may be eliminated in an apparatus and method in accordance with the invention. Meanwhile, observation to determine whether leakage from veins, swelling, inflammation, kinked tubing, infection, collection of fluids, weeping of fluids, and the like have occurred is readily visible.

The present invention may be embodied in other specific forms without departing from its spirit or essential characteristics. The described embodiments are to be considered in all respects only as illustrative, and not restrictive. The scope of the invention is, therefore, indicated by the appended claims, rather than by the foregoing description. All changes which come within the meaning and range of equivalency of the claims are to be embraced within their scope.

What is claimed and desired to be secured by United States Letters Patent is:

1. An apparatus comprising:
an intravenous supply comprising a container holding a fluid, an adapter to conduct the fluid from the container to a line; the line connected to feed the fluid to a hub, and the hub sized and shaped to receive a catheter sealing securable thereto to receive the fluid;
the catheter sized to provide intravenous introduction of the fluid, the catheter comprising a tubular portion placed into a vein, a base fixedly secured to the tubular portion to sealingly engage the hub and deliver the fluid to the tubular portion to be discharged into a vein;
a needle temporarily insertable through the tubular portion to render the tubular portion mechanically rigid during insertion into a vein of a subject by a user, the needle comprising a point extending beyond a distal end of the tubular portion to engage and penetrate a vein during insertion thereinto of the tubular portion;
a mount having a resilience selected to distribute stresses imposed by at least one of the base and the hub, the mount being positioned between the skin of a subject and at least one of the base and the hub.

2. The apparatus of claim 1, wherein the mount further comprises an upper surface and a lower surface, the lower surface being provided with a layer of adhesive to secure the lower surface to the skin of a subject receiving the catheter.

3. The apparatus of claim 2, further comprising a securement member comprising a layer effective to urge at least one of the base and hub toward the skin of a subject.

4. The apparatus of claim 3, wherein the securement member is transparent to render the catheter and surrounding skin of a subject visible therethrough for visible inspection by a user.

5. The apparatus of claim 4, wherein the mount is sized to maintain at least one of the base and hub away from contact with the skin of a subject, distributing across the lower surface of the mount the load imposed by at least one of the base and hub on the upper surface thereof.

6. The apparatus of claim 5, wherein the mount is formed of a polymer.

7. The apparatus of claim 6, wherein the polymer is expanded polymer to form a foam.

8. The apparatus of claim 7, wherein the expanded polymer is a closed cell foam.

9. The apparatus of claim 1, wherein the mount is shaped to provide relief to receive at least one of the base and hub thereinto to stabilize the catheter against movement with respect to the mount and the skin of the subject.

10. The apparatus of claim 1, wherein the catheter is positioned in an inserted condition in a vein proximate the skull of a subject, and wherein the upper surface of the mount is formed to orient at least one of the base and hub to stabilize the base and hub to extend substantially tangentially away from the skull.

11. The apparatus of claim 1, further comprising a securement member urging at least one of the base and hub against the mount, the securement member having a transparent portion to support visible inspection of the catheter and surrounding skin of a subject.

12. A method comprising:
providing a catheter to be in fluid communication with blood in a vein of a subject, the catheter comprising a tubular portion and a base, the base being secured to the tubular portion and sized to receive a needle passing through the base and into the tubular portion;
providing a hub secured to the base to feed a fluid therethrough to the tubular portion;
selecting a site for insertion of the tubular portion into a vein of a subject;
providing a mount sized to correspond to a tangent to a surface of the skin of a subject proximate a vascular site receiving the catheter;
threading a needle through the tubular portion to render the tubular portion rigid and to expose a point of the needle for insertion into a vein of a subject;
inserting the needle into the vein;
withdrawing the needle, leaving in the vein the tubular portion;
positioning the mount between the skin of a user and at least one of the hub and the base to position the hub and base to extend substantially tangentially away from the site; and
securing the catheter, hub, and mount proximate the site to move therewith without substantial relative motion therebetween.

13. The method of claim 12, further comprising providing a securement member having a transparent portion rendering the catheter and skin at the site available for visual inspection by a user.

14. The method of claim 12, further comprising providing an intravenous supply comprising a container holding a fluid, an adapter to conduct the fluid from the container to a line; the line connected to feed the fluid to the hub.

15. The method of claim 12, wherein the mount is formed of a polymer selected to have a resilience effective to distribute stresses imposed by at least one of the base and the hub to the skin at a reduced value over an increased area of the skin.

16. The method of claim 12, wherein the mount further comprises an upper surface and a lower surface, the lower surface being provided with a layer of adhesive to secure the lower surface to the skin of the subject.

17. The method of claim 12, further comprising sizing the mount to maintain at least one of the base and hub away from contact with the skin of a subject, distributing across a lower surface of the mount a load imposed by at least one of the base and hub on the upper surface thereof.

18. The method of claim 12, wherein the mount is formed of an expanded polymer in a closed-cell foam.

19. The method of claim 12, further comprising:
positioning the catheter in an inserted condition in a vein proximate the skull of a subject; and
positioning the mount to orient at least one of the base and hub to stabilize the base and hub to extend substantially tangentially away from the skull.

20. A method to relieve stress concentrations and stabilize a catheter in an infant, the method comprising:
providing a catheter to catheterize a vascular site of a subject;
providing a supply comprising a line to feed a liquid and a hub to sealingly engage the catheter;
installing the catheter in a vein of a subject at the site;
positioning a closed-cell, foam mount between the skin of the subject and the catheter to distribute stresses otherwise applied to the skin by at least one of the catheter and the hub; and
securing the catheter, hub, and mount to the skin of a user, by applying a covering urging the catheter and hub toward the mount, and stabilizing the mount and catheter in at least one direction orthogonal thereto.
